(19) United States
(12) Patent Application Publication
(10) Pub. No.: US 2020/0401841 A1
(43) Pub. Date: Dec. 24, 2020

(54) APPARATUS FOR DIAGNOSING GLAUCOMA

(71) Applicant: SAMSUNG SDS CO., LTD., Seoul (KR)

(72) Inventors: JoonSeok Lee, Seoul (KR); JoonHo Lee, Seoul (KR); MinYoung Lee, Seoul (KR); JiEun Song, Seoul (KR); SooAh Cho, Seoul (KR)

(21) Appl. No.: 16/664,834

(22) Filed: Oct. 26, 2019

(30) Foreign Application Priority Data
Jun. 24, 2019 (KR) 10-2019-0074752

(51) Int. Cl. G06K 9/62; A61B 3/12; A61B 5/00; G06T 7/00

(52) U.S. Cl. CPC G06K 9/6202; A61B 3/12; G06K 2009/6213; G06T 7/0014; A61B 5/7267

(57) ABSTRACT

A glaucoma diagnosis apparatus according to an embodiment includes a fundus image processor configured to receive a fundus image and extract a first region of interest (ROI) and a second ROI from the received fundus image, an image classification neural network configured to learn the extracted first ROI and perform classification into a normal fundus image and a glaucoma fundus image on the basis of the learned first ROI, a vertical cup-to-disc ratio (vCDR) calculator configured to recognize an optic disc (OD) and an optic cup (OC) from the extracted second ROI and calculate a vCDR, and a determinator configured to aggregate a vCDR calculation result and an image classification result of the image classification neural network to determine whether glaucoma is present in the fundus image.

### TECHNICAL FIELD

Embodiments of present invention relate to fundus image processing and a glaucoma diagnosis technique using the same.

### BACKGROUND ART

Glaucoma is a disease in which optic nerves gradually and chronically damaged by elevation of intraocular pressure result in a visual field defect. Glaucoma is one representative eye disease that causes blindness, but due to lack of eye specialists, glaucoma is often not diagnosed on time. Thus, recently, a technique for diagnosing glaucoma using machine learning, especially deep learning, has been proposed.

In order to increase the accuracy of machine learning-based glaucoma diagnosis, it is necessary to learn a large number of fundus images. However, fundus images can only be acquired by examination, and labeling according to a diagnosis by a specialist is essential. Thus, there is a difficulty in obtaining a large amount of high-quality data. Also, machine learning-based prediction models have a limitation in that the basis of a prediction result is very difficult or impossible to explain (inexplicable).

### DISCLOSURE

### Technical Problem

Embodiments of the present disclosure are directed to providing a technical means for effectively diagnosing glaucoma using fundus images.

### Technical Solution

According to an aspect of the present disclosure, there is provided a glaucoma diagnosis apparatus including a fundus image processor configured to receive a fundus image and extract a first region of interest (ROI) and a second ROI from the received fundus image, an image classification neural network configured to learn the extracted first ROI and perform classification into a normal fundus image and a glaucoma fundus image on the basis of the learned first ROI, a vertical cup-to-disc ratio (vCDR) calculator configured to recognize an optic disc (OD) and an optic cup (OC) from the extracted second ROI and calculate a vCDR, and a determinator configured to aggregate a vCDR calculation result and an image classification result of the image classification neural network to determine whether glaucoma is present in the fundus image.

The fundus image processor may include an optic disc detection module configured to detect an optic disc from the received fundus image; an image rotation module configured to rotate the fundus image around center coordinates of the optic disc such that a slope between the center coordinates of the optic disc and a center of the fundus image is constant; a first ROI extraction module configured to extract the first ROI from the rotated fundus image such that the detected optic disc is placed at an upper left end or an upper right end of the first ROI; and a second ROI extraction module configured to extract the second ROI from the received fundus image such that the detected optic disc is placed at the center of the second ROI.

The optic disc detection module may detect the optic disc using an image obtained through a polar coordinate transformation on the fundus image.

The fundus image processor may further include a preprocessing module configured to resize the fundus image to a predetermined size before the optic disc is detected and horizontally flip the fundus image depending on whether the fundus image is a right fundus image or a left fundus image.

The image rotation module may set the center of the fundus image as an origin of a coordinate plane, calculate an angle between the center coordinates of the detected optic disc and a horizontal axis (an x-axis) of the coordinate plane, and rotate the fundus image around the origin such that the angle matches a predetermined reference angle.

The reference angle may be any one of 45 degrees or 135 degrees.

The image rotation module may augment the number of fundus images by flipping the fundus image according to a reference line connecting the origin of the rotated fundus image and the center coordinates of the optic disc or by additionally rotating the rotated fundus image within a predetermined additional rotation range around the origin of the rotated fundus image.

The first ROI extraction module may extract the ROI such that the center coordinates of the optic disc are spaced one-quarter of a side length from an upper left end or an upper right end of the first ROI.

The fundus image processor may further include a histogram matching module configured to, when the fundus image is a test image of a machine learning model, perform histogram matching on the first ROI of the test image on the basis of an average histogram of images included in a learning dataset of the machine learning model.

The vCDR calculator may include a first mask generation module configured to generate a first mask corresponding to the optic disc using an image obtained through a polar coordinate transformation on the second ROI; a second mask generation module configured to extract a sub-region corresponding to the first mask from the second ROI and generate a second mask corresponding to the optic cup from the extracted sub-region; and a calculation module configured to overlay the first mask and the second mask and calculate the vCDR.

The first mask generation module may augment the number of second ROIs by horizontally or vertically moving the center of the second ROI within a predetermined range or by horizontally shifting the image obtained through the polar coordinate transformation on the second ROI within a predetermined range.

The calculation module may calculate the vCDR using a result of combining the first mask and the second mask generated from an additional image generated by augmenting the same second ROI.

The determinator may determine whether glaucoma is present in the fundus image through logistic recession analysis on the image classification result and the vCDR calculation result.

The determinator may perform the logistic regression analysis by using first- to nth-order terms (here, n is a natural number of 2 or more) of the vCDR calculation result as an input value in addition to the image classification result.

### Effects of the Invention

According to the disclosed embodiments, by efficiently setting a region of interest (ROI) of a fundus image, it is possible to effectively increase the accuracy of glaucoma classification in a glaucoma diagnosis field where the acquisition of an image for machine learning is limited.

Also, according to the disclosed embodiments, by diagnosing glaucoma by utilizing a vCDR in addition to a classification result using machine learning, it is possible to partially compensate for inexplicability, which is one disadvantage of a deep learning-based classifier, and it is also possible to improve final classification performance, thereby achieving high performance with a very small amount of data.

### BRIEF DESCRIPTION OF THE DRAWINGS

FIG. 1 is a block diagram illustrating a glaucoma diagnosis apparatus 100 according to an embodiment;

FIG. 2 is a block diagram illustrating a detailed configuration of a fundus image processor 102 according to an embodiment;

FIGS. 3 and 4 are flowcharts illustrating a fundus image processing process of the fundus image processor 102 according to an embodiment;

FIG. 5 is an exemplary diagram illustrating the position of an optic disc observed in a fundus image and a position where retinal nerve fiber layer defects (RNFLD) are mainly observed in an optic nerve;

FIG. 6 is an exemplary diagram illustrating a process of detecting an optic disc (OD) from a fundus image by means of an optic disc detection module 204 according to an embodiment;

FIG. 7 is an exemplary diagram showing a change in a fundus image according to a camera's features;

FIG. 8 is an exemplary diagram illustrating a histogram matching process of a histogram matching module of the fundus image processor 102 according to an embodiment;

FIG. 9 is a block diagram illustrating a detailed configuration of a vertical cup-to-disc ratio (vCDR) calculator 106 according to an embodiment;

FIG. 10 is a flowchart illustrating a fundus image processing process of the vCDR calculator 106 according to an embodiment;

FIG. 11 is an exemplary diagram showing an example of augmenting the number of second regions of interest (ROIs) by horizontally or vertically moving the center of a second ROI;

FIG. 12 is an exemplary diagram showing an example of augmenting the number of second ROIs by horizontally shifting an image obtained through a polar coordinate transformation on a second ROI within a predetermined range;

FIG. 13 is an exemplary diagram showing an example of a calculation module 906 combining a first mask and a second mask obtained by augmenting the same second ROI;

FIG. 14 is an exemplary diagram illustrating a process of determining whether glaucoma is present in an image by means of a determinator 108 according to an embodiment; and

FIG. 15 is a block diagram illustrating a computing environment 10 including a computing apparatus suitable for use in example embodiments.

### DETAILED DESCRIPTION

Hereinafter, embodiments of the present disclosure will be described in detail with reference to the accompanying drawings. The following detailed description is provided to assist the reader in gaining a comprehensive understanding of methods, apparatuses, and/or systems described herein. However, the description is only an example, and the present disclosure is not limited thereto.

In describing the embodiments of the present disclosure, when it is determined that a detailed description of a known technique associated with the present disclosure would unnecessarily obscure the subject matter of the present disclosure, the detailed description will be omitted. Also, terms used herein are defined in consideration of the functions of the present disclosure and may be changed depending on a user, the intent of an operator, or a custom. Therefore, the definition should be made based on the contents throughout the specification. The terminology used herein is only for the purpose of describing embodiments of the present disclosure and is not restrictive. The singular forms “a,” “an,” and “the” are intended to include the plural forms as well, unless the context clearly indicates otherwise. It should be understood that the terms “comprises,” “comprising,” “includes,” and/or “including” specify the presence of stated features, integers, steps, operations, elements, components, and/or groups thereof when used herein but do not preclude the presence or addition of one or more other features, integers, steps, operations, elements, components, and/or groups thereof.

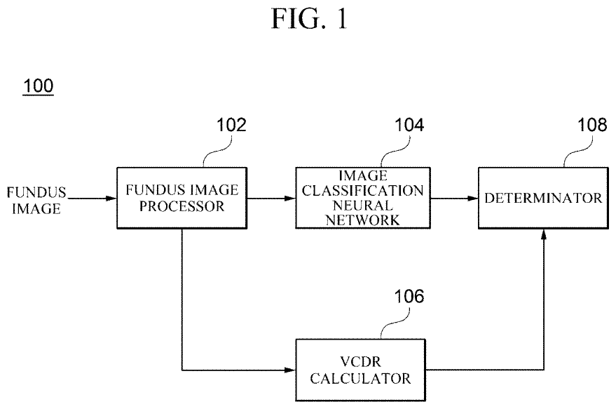

FIG. 1 is a block diagram illustrating a glaucoma diagnosis apparatus 100 according to an embodiment. The glaucoma diagnosis apparatus 100 according to an embodiment is an apparatus for receiving a fundus image, analyzing the received fundus image, and determining whether glaucoma has developed. As shown, the glaucoma diagnosis apparatus 100 according to an embodiment includes a fundus image processor 102, an image classification neural network 104, a vertical cup-to-disc ratio (vCDR) calculator 106, and a determinator 108.

The fundus image processor 102 receives a fundus image and converts the received fundus image into a form suitable for training the image classification neural network 104. In an embodiment, the fundus image processor 102 is configured to convert a fundus image through a process of resizing, rotation, and ROI setting related to the fundus image and is configured to extract a first ROI and a second ROI.

The image classification neural network 104 is a machine learning-based neural network configured to learn the first ROI of the fundus image extracted by the fundus image processor 102 and perform classification into a normal fundus image and a glaucoma fundus image on the basis of the learned first ROI. In an embodiment, the image classification neural network 104 may have various structures in consideration of the features of the fundus image. For example, the image classification neural network may be a convolutional neural network (CNN)-based neural network including one or more convolution layers and one or more pooling layers. However, this is an example, and thus the image classification neural network 104 may have any structure as needed and is not limited to a specific machine learning model.

The vCDR calculator 106 detects an optic cup (OC) and an optic disc (OD) from the second ROI of the fundus image extracted by the fundus image processor 102 and calculates a vCDR on the basis of the detected OC and OD. The most common cause of glaucoma is intraocular pressure elevation. When optic nerves are damaged due to the intraocular pressure elevation, the OC increases in size, and the vCDR increases. Therefore, the vCDR is a very important factor for clinically diagnosing glaucoma. To this end, the vCDR calculator 106 is configured to compensate for a limitation in accuracy due to a small dataset size by calculating the vCDR separately from the image classification neural network 104 and utilizing the vCDR to determine glaucoma.

The determinator 108 may aggregate the classification result of the image classification neural network 104 and the vCDR calculation result of the vCDR calculator 106 to determine whether glaucoma is present in a fundus image. In an embodiment, the determinator 108 may determine whether glaucoma is present in a fundus image by using regression analysis on the image classification result and the vCDR calculation result. For example, the determinator 108 may utilize logistic regression as a regression analysis model. Also, the determinator 108 may use multiple-order terms of the calculated vCDR as an input of the regression analysis model in order to avoid the linearity of input values. In detail, the determinator 108 may perform the logistic regression analysis by using first- to nth-order terms (here, n is a natural number of two or more) of the vCDR calculation result as an input value. This will be described in detail later. In this way, when the vCDR is utilized together with the classification result of the image classification neural network 104, it is possible to partially compensate for inexplicability, which is one disadvantage of a deep learning-based classifier, and it is also possible to improve final classification performance, thereby achieving high performance with a very small amount of data.

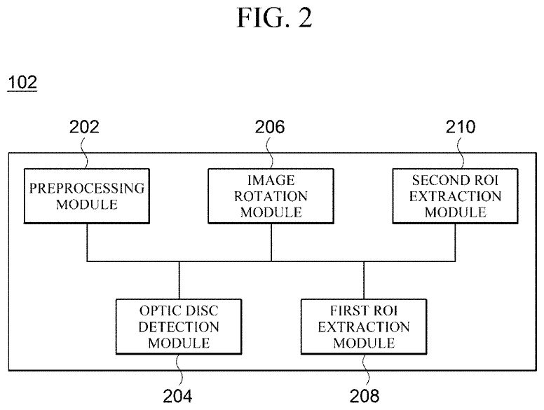

FIG. 2 is a block diagram illustrating a detailed configuration of a fundus image processor 102 according to an embodiment. As shown, the fundus image processor 102 according to an embodiment includes a preprocessing module 202, an optic disc detection module 204, an image rotation module 206, a first ROI extraction module 208, and a second ROI extraction module 210.

The preprocessing module 202 resizes an input fundus image to a predetermined size and horizontally flips the fundus image depending on whether the fundus image is a right fundus image or a left fundus image.

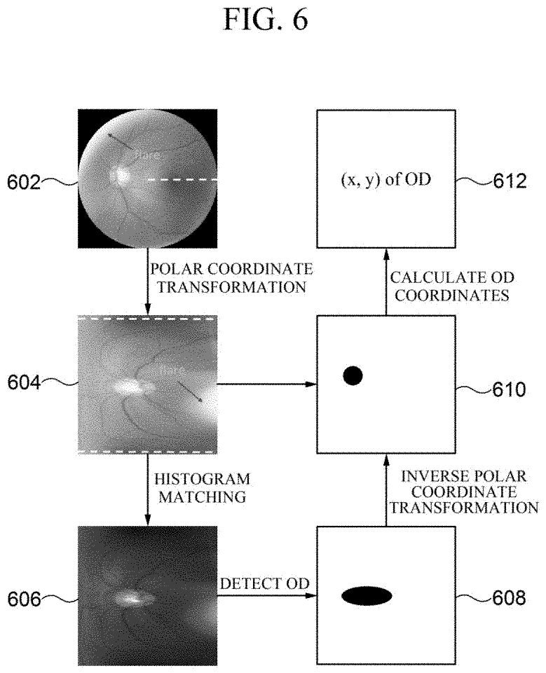

The optic disc detection module 204 may detect an optic disc (OD) from the input fundus image. The optic disc is an exit point for ganglion cell axons coming together and then leaving the eye and is also an entry point for major blood vessels that supply nutrition to the retina. The optic disc is generally placed 3 to 4 mm from the yellow spot to the nasal side. The optic cup (OC), which is cup-like in shape, is inside the optic disc. In an embodiment, the optic disc detection module 204 may detect the optic disc using a separate image segmentation neural network or the like and may detect the optic cup (OC) in addition to the optic disc if necessary. A configuration for detecting an optic disc from a fundus image will be described in detail below with reference to FIG. 6.

The image rotation module 206 may rotate the fundus image with respect to the center coordinates such that the slope between the center of the fundus image and the center coordinates of the optic disc is constant for each fundus image. In detail, the image rotation module 206 may be configured to set the center of the fundus image as the origin of the coordinate plane, calculate an angle between the center coordinates of the detected optic disc and the horizontal axis (the x-axis) of the coordinate plane, and rotate the fundus image around the origin such that the angle matches a predetermined reference angle.

The first ROI extraction module 208 extracts an ROI from the fundus image rotated by the image rotation module 206 in consideration of the position of the optic disc in the rotated fundus image. In detail, the first ROI extraction module 208 extracts a first ROI from the rotated fundus image such that the detected optic disc is placed at an upper left end or an upper right end of the first ROI.

The second ROI extraction module 210 extracts a second ROI from the input fundus image such that the optic disc detected by the optic disc detection module 204 is placed in the center of the second ROI. The second ROI extraction module 208 is different from the first ROI extraction module 208 in that an original image is used rather than the rotated image and also in that an ROI is extracted such that the optic disc is placed at the center.

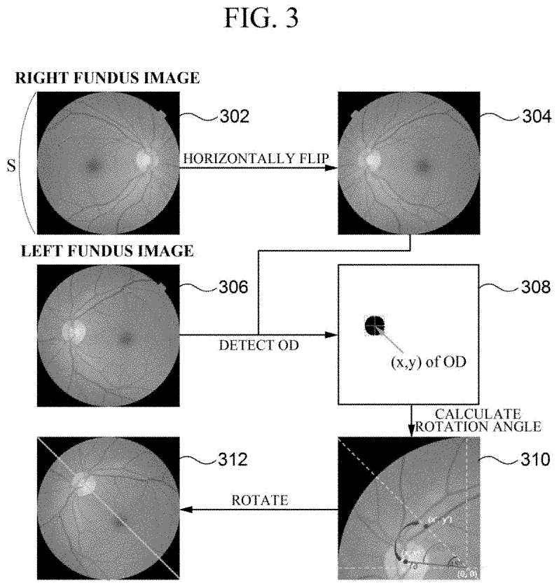

FIG. 3 is a flowchart illustrating a fundus image processing process in the fundus image processor 102 according to an embodiment.

When a fundus image is input to the fundus image processor 102, first, the preprocessing module 202 resizes the fundus image to the same size as those of other fundus images (not shown). For example, the preprocessing module 202 may adjust input fungus images in size such that the fungus images are in the shape of a square with a side having a length of S. In this process, as shown in FIG. 3, the preprocessing module 202 may adjust the position of a fundus in the fundus image or crop a portion of the fundus image such that an outer diameter of the circular fundus touch each side of the square.

Subsequently, the preprocessing module 202 horizontally flips the fundus image depending on whether the fundus image is a right fundus image or a left fundus image. In the shown embodiment, the input fundus image is horizontally flipped like an image 304 when the input fundus image is a right fundus image 302, and the input fundus image is used without change when the input fundus image is a left fundus image 306. Generally, since a left fundus image and a right fundus image have symmetry, the same process may be applied irrespective of whether an input fundus image is a left fundus image or a right fundus image by horizontally flipping the image depending on whether the image is a left fundus image or a right fundus image.

Subsequently, the optic disc detection module 204 detects an optic disc from the fully preprocessed fundus image. As shown in FIG. 3, an image 308 indicates the position of the optic disc detected from the fundus image 304 or 306 (a black point depicted in the drawing corresponding to the optic disc).

Subsequently, the image rotation module 206 may rotate the fundus image with respect to the center coordinates such that the slope between the center of the fundus image and the center coordinates of the optic disc is constant for each fundus image.

In detail, the image rotation module 206 sets the center of the fundus image as the origin of the coordinate plane as shown in an image 310 and calculates an angle ∂ between the center coordinates of the detected optic disc and the horizontal axis (the x-axis) of the coordinate plane.

Subsequently, the image rotation module 206 rotates the entire fundus image with respect to the origin such that the angle matches a predetermined reference angle. In an embodiment, the reference angle may be any one of 45 degrees or 135 degrees. For example, when the reference angle is set to 135 degrees, an image in which the center of the optic disc is present on a straight line y=−x may be obtained. For example, when the reference angle is set to 45 degrees, an image in which the center of the optic disc is present on a straight line y=x may be obtained. An image 312 in FIG. 3 is rotated such that the center of the optic disc detected from the fundus image is present on the straight line y=−x.

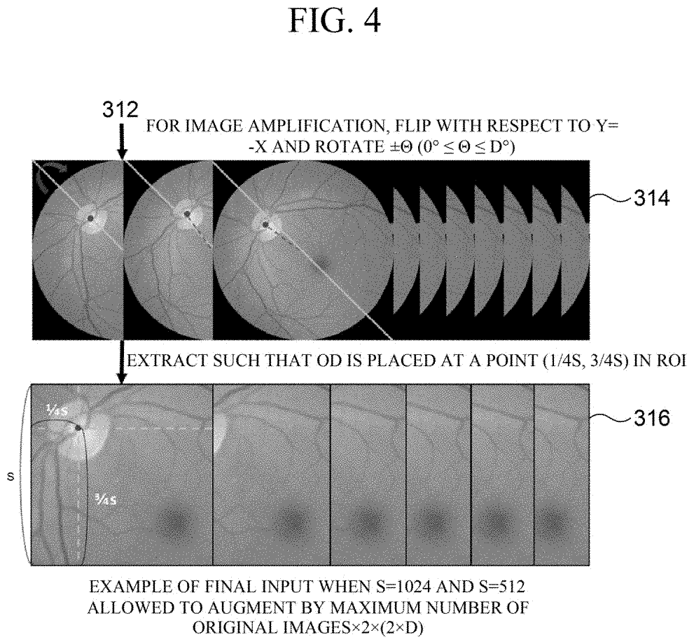

In an embodiment, the image rotation module 206 may utilize limited rotation and flipping of a rotated image as shown in 314 of FIG. 4 in order to augment the number of pieces of fundus image data.

For example, the image rotation module 206 may flip the fundus image along a reference line connecting the origin of the rotated fundus image and the center coordinates of the optic disc. Also, the image rotation module 206 may further rotate the rotated fundus image within an additional rotation range (±θ, 0°≤θ≤D°) preset with respect to the origin of the rotated fundus image. The reason why the reference line is used and the rotation angle is limited when the fundus image is flipped is to allow a prediction model to learn not only characteristic patterns of a glaucoma image but also the absolute position of each pattern.

Through the flipping or additional rotation of an image, the image rotation module 206 may have an effect such as increasing the number of images to be learned by the image classification neural network 104. In detail, through the flipping of a fundus image, the image rotation module 206 may obtain a data augmentation effect. That is, the image rotation module 206 may augment the number of fundus images by a factor of two. Also, through the additional rotation of a fundus image, the image rotation module 206 may obtain a data augmentation effect. That is, the image rotation module 206 may augment the number of fundus images by a factor of 2×D. That is, when the flipping and additional rotation of fundus images is used, n fundus images may be augmented by a factor of n×2×2×D.

Subsequently, the first ROI extraction module 208 extracts a first ROI from the rotated fundus image in consideration of the position of the optic disc in the fundus image rotated by the image rotation module 206. The image designated by “316” in FIG. 3 indicates a first ROI extracted from an augmented fundus image 314. In an embodiment, the first ROI extraction module 208 may extract the ROI such that the center coordinates of the optic disc are placed at a predetermined specific point of the first ROI, for example, such that the center coordinates of the optic disc are spaced one-quarter of the side length from an upper left end or an upper right end of the first ROI. For example, as shown in FIG. 4, the first ROI extraction module 208 may set an ROI in the fundus image such that the center coordinates of the optic disc are spaced one-quarter of the diagonal length from the upper left end of the first ROI (i.e., such that, when the length of one side of the ROI is “S,” the center coordinates of the optic disc are placed at (¼S, ¾S)). Also, the first ROI extraction module 208 may be configured to set an ROI in consideration of the size of the ROI in the fundus image, the degree to which a region surrounding the optic disc is included, etc. FIGS. 3 and 4 illustrate an example in which one side of an initially input fundus image has a length S of 1024 pixels and one side of an ROI has a length S of 512 pixels.

Last, the second ROI extraction module 210 extracts a certain region placed around the optic disc detected by the optic disc detection module 204 from the fundus image as a second ROI. The second ROI extraction module 210 may extract the second ROI such that the optic disc is sufficiently included in the second ROI, but the degree of inclusion of an optic disc periphery may be appropriately set in consideration of the features of the fundus image.

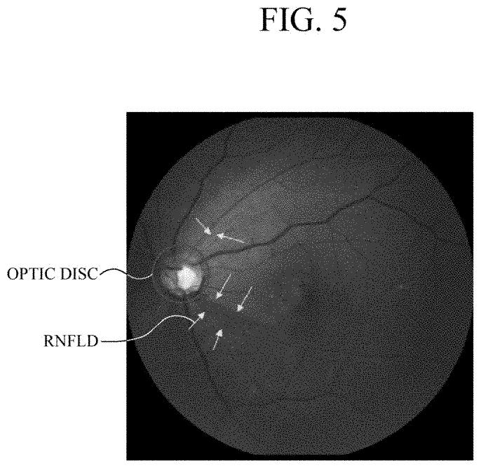

FIG. 5 is an exemplary diagram illustrating the position of an optic disc observed in a fundus image and a position where retinal nerve fiber layer defects (RNFLD) are mainly observed in an optic nerve. Optic nerve damage and RNFLD, which are major lesions of glaucoma, tend to concentrate mainly around the optic disc. Accordingly, in the embodiments of the present disclosure, the fundus image processor 102 is configured to concentrate the first ROI as much as possible in the range where the major lesions of glaucoma mainly occur. Thus, it is possible to minimize the reduction of a high-resolution fundus image such that the high-resolution fundus image may be used as an input of the image classification neural network 104 without great loss of the image. Also, the fundus image processor 102 is configured to rotate fundus images such that the optic disc is present at a certain position for each fundus image. Thus, the image classification neural network 104 may train the pattern of glaucoma and also the absolute position of the pattern.

FIG. 6 is an exemplary diagram illustrating a process of detecting an optic disc (OD) from a fundus image using an optic disc detection module 204 according to an embodiment. In an embodiment, the optic disc detection module 204 may be configured to detect the optic disc using an image obtained through a polar coordinate transformation on a fundus image. This will be described in more detail as follows.

First, the optic disc detection module 204 performs a polar coordinate transformation on a fundus image 602 on the basis of a straight line (depicted as a dotted line in the drawing) connecting from the center of the fundus image 602 to an edge thereof. Here, the polar coordinate transformation means that the circular fundus image 602 is spread on the basis of the straight line and transformed into a rectangular image. In FIG. 6, an image on which a polar coordinate transformation has been performed is designated by 604. That is, dotted lines shown in the fundus image 602 and the polar coordinate-transformed image 604 indicate the same part in the fundus.

The reason for the polar coordinate transformation on the fundus image like this is to prevent a decrease in the accuracy of detection of the optic disc due to a flare phenomenon appearing at outer portions of some fundus images. As shown in 602 of FIG. 6, a flare usually appears in an outer part of a fundus image. When a polar coordinate transformation is performed on a fundus image, the area of the outer portion of the fundus image becomes relatively narrow as shown in 604. As a result, it is possible to minimize the effect due to the flare.

Subsequently, if necessary, the optic disc detection module 204 allows an optic disc part to be highlighted in the fundus image through histogram matching on the image on which the polar coordinate transformation has been performed. A portion 606 of FIG. 6 illustrates an image obtained through histogram matching on the image on which the polar coordinate transformation has been performed.

Subsequently, the optic disc detection module 204 detects an optic disc from the image on which the polar coordinate transformation has been performed. A portion 608 of FIG. 6 illustrates the position of an optic disc in the image on which the polar coordinate transformation has been performed and which is detected by the optic disc detection module 204.

Subsequently, the optic disc detection module 204 acquires the position of the optic disc from the original fundus image through an inverse polar coordinate transformation on the position where the optic disc is detected. In the disclosed embodiments, the inverse polar coordinate transformation refers to the inverse process for the above polar coordinate transformation. A portion 610 of FIG. 6 indicates the position of the optic disc acquired through the inverse polar coordinate transformation.

Last, the optic disc detection module 204 calculates the center point (x, y) of the acquired optic disc (612).

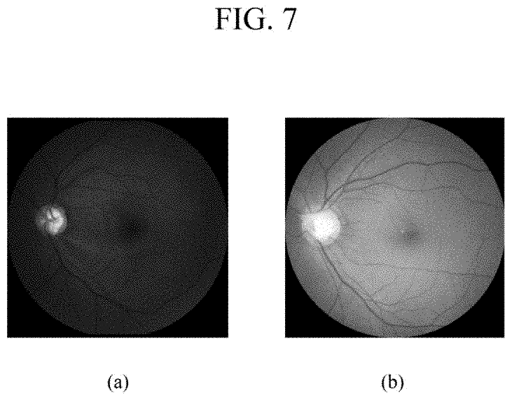

Meanwhile, the fundus image processor 102 according to an embodiment may further include a histogram matching module (not shown). Since the fundus image is captured using a digital camera, the features of the image differ depending on the camera manufacturer, the camera model, and the like. FIG. 7 is an exemplary diagram showing a change in a fundus image according to a camera's features. As shown, it can be seen that image (a) and image (b) captured using different cameras differ in terms of color, brightness, and the like. In order to overcome the difference of image features caused depending on the camera, a histogram matching technique may be utilized in the disclosed embodiments. The histogram matching is an image pre-processing technique for matching the histogram of a source image to the histogram of a target image.

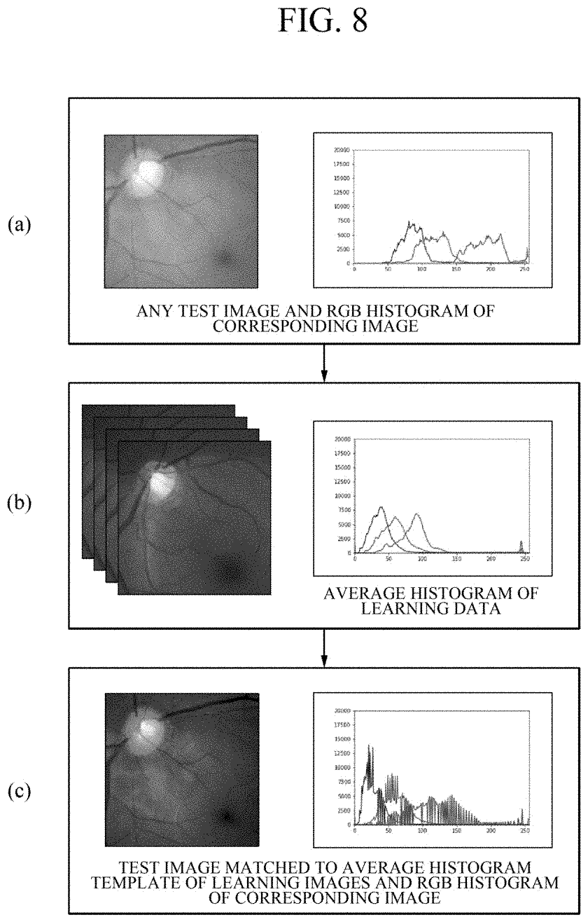

FIG. 8 is an exemplary diagram illustrating a histogram matching process of a histogram matching module of the fundus image processor 102 according to an embodiment. When an input fundus image is a test image of a machine learning model, the histogram matching module may perform histogram matching on an ROI of the test image on the basis of an average histogram included in a learning data set of the machine learning model. In the exemplary diagram illustrated in FIG. 8, portion (a) shows an input test image (left) and an RGB histogram of the corresponding image (right), portion (b) shows learning data of the image classification neural network 104 (left) and an average RGB histogram of the corresponding learning data (right), and portion (c) show a test image when the histogram of portion (a) is matched to the histogram of portion (b) (left) and an RGB histogram of the corresponding image (right). In this case, the average RGB histogram of the learning data may be calculated by the histogram matching module during the preprocessing process for the learning data and then stored.

The image classification neural network 104 may have reduced performance stability for new data placed outside the distribution range of the learned data. Also, the distribution of data to be tested in the future cannot be determined at the time of training the neural network because the distribution may approach infinity. Thus, according to the present disclosure, through the above histogram matching, it is possible to compensate for the narrowness of the distribution range of the neural network caused by a small learning dataset.

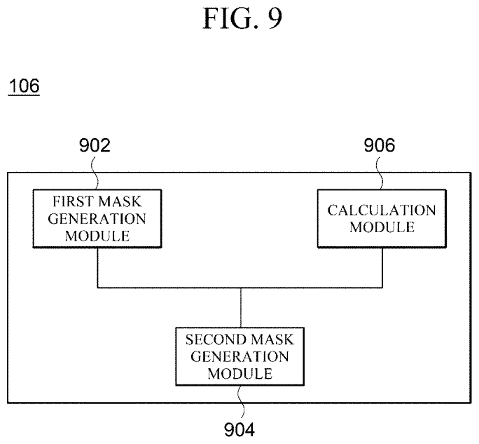

FIG. 9 is a block diagram illustrating a detailed configuration of a vertical cup-to-disc ratio (vCDR) calculator 106 according to an embodiment. As shown, the vCDR calculator 106 according to an embodiment includes a first mask generation module 902, a second mask generation module 904, and a calculation module 906.

The first mask generation module 902 generates a first mask corresponding to the optic disc using an image obtained through a polar coordinate transformation on the second ROI generated by the second ROI extraction module 210.

The second mask generation module 904 extracts a sub-region corresponding to the first mask from the second ROI and generates a second mask corresponding to the optic cup from the extracted sub-region.

The calculation module 906 calculates the vCDR by overlaying the first mask and the second mask.

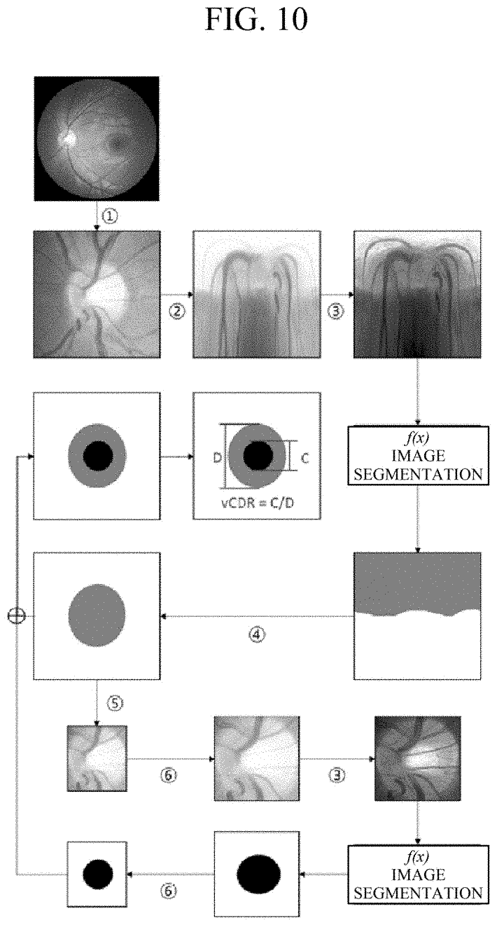

FIG. 10 is a flowchart illustrating a fundus image processing process in the vCDR calculator 106 according to an embodiment.

First, when a second ROI 1004 is generated from a fundus image 1002, the first mask generation module 902 applies a polar coordinate transformation to the second ROI to obtain an image such as 1006. The polar coordinate transformation on the image has been described in detail with reference to 6, and thus a redundant description thereof will be omitted. Subsequently, the first mask generation module 902 applies histogram matching to the image 1006 on which the polar coordination transformation has been performed.

The first mask generation module 902 may perform the histogram matching using an average histogram of a learning image of an image segmentation neural network 1010 to be described below. In FIG. 10, an image 1008 indicates an image obtained by applying histogram matching to the image 1006.

Subsequently, the first mask generation module 902 segments the image 1008, which is obtained through histogram transformation using the image segmentation neural network 1010, into pixels corresponding to the optic disc and pixels not corresponding to the optic disc. In an image 1012, a part masked in gray is a pixel corresponding to the optic disc, and a part masked in white is a pixel not corresponding to the optic disc. In an embodiment, the image segmentation neural network 1010 is a neural network configured to learn an image including the optic disc and discover a pixel corresponding to the optic disc from a given image. Subsequently, the first mask generation module 902 applies an inverse polar coordinate transformation to a segmentation result 1012 to obtain a first mask corresponding to the optic disc. In FIG. 10, an image 1014 corresponds to the first mask.

Meanwhile, the second mask generation module 904 extracts a sub-region 1016 corresponding to the first mask 1014 from the second ROI 1004. Subsequently, the second mask generation module 904 resizes the extracted sub-region 1016 (1018) and applies histogram matching (1020). Like the first mask generation module 902, the second mask generation module 904 may also perform the histogram matching using an average histogram of a learning image of the image segmentation neural network 1022.

Subsequently, the second mask generation module 904 segments the image 1020, which is obtained through histogram transformation using the image segmentation neural network 1022, into pixels corresponding to the optic cup (OC) and pixels not corresponding to the optic cup. In an image 1024, a part masked in black is a pixel corresponding to the optic cup, and a part masked in white is a pixel not corresponding to the optic cup. In an embodiment, the image segmentation neural network 1022 is a neural network configured to learn an image including the optic cup and discover a pixel corresponding to the optic cup from a given image. Subsequently, the second mask generation module 904 resizes the segmentation result 1024 to the same size as the sub-region 1016 to obtain a second size 1026. The second mask generation module 904 does not perform a polar coordinate transformation on the sub-region 1016, unlike the first mask generation module 902. This is because, when the vCDR is very large, a region corresponding to the optic cup in the sub-region 106 may be lost during the polar coordinate transformation.

Last, the calculation module 906 overlays the first mask and the second mask as shown in 1028 and then calculates the vCDR on the basis of the overlay (1030).

In an embodiment, the first mask generation module 902 may augment the number of second ROIs by horizontally or vertically moving the center of the second ROI within a predetermined range or by horizontally shifting an image obtained through a polar coordinate transformation on the second ROI within a predetermined range.

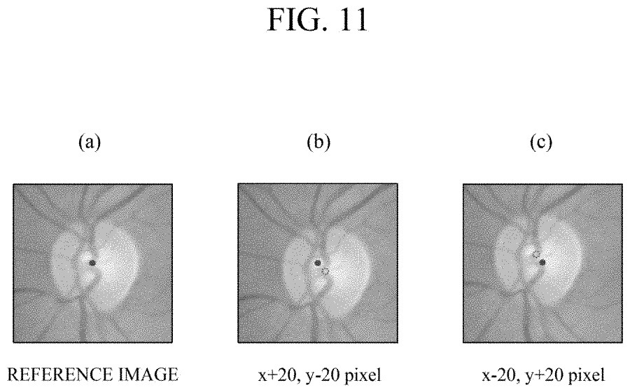

FIG. 11 is an exemplary diagram showing an example of augmenting the number of second ROIs by horizontally or vertically moving the center of the second ROI. In detail, FIG. 11(a) shows an original image of a second ROI, FIG. 11(b) shows an image obtained by moving the image shown in FIG. 11(a) by +20 pixels in the x-direction and by −20 pixels in the y-direction, and FIG. 11(c) shows an image obtained by moving the image shown in FIG. 11(a) by −20 pixels in the x-direction and by +20 pixels in the y-direction. When the center adjustment value for augmenting the second ROI is (±Δx, ±Δy) and the ranges of Δx and Δy are limited to d, an image augmented by a factor of n×2×d×2×D may be obtained for the number n of original images (second ROIs).

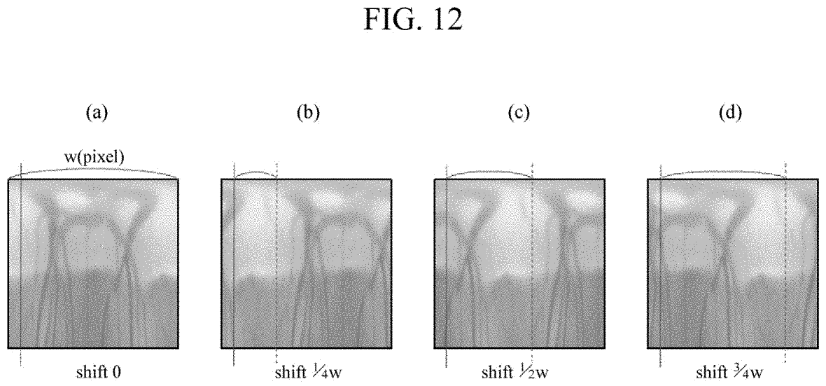

FIG. 12 is an exemplary diagram showing an example of augmenting the number of second ROIs by horizontally shifting an image obtained through a polar coordinate transformation on a second ROI within a predetermined range. In detail, FIG. 12(a) shows an original image of a second ROI on which a polar coordinate transformation has been performed, FIG. 12(b) shows an image obtained by shifting the image shown in FIG. 12(a) to the right by ¼th of the width, FIG. 12(c) shows an image obtained by shifting the image shown in FIG. 12(a) to the right by half of the width, and FIG. 12(d) shows an image obtained by shifting the image shown in FIG. 12(a) to the right by ¾th of the width. When the image is shifted to the right, a portion placed outside the range of the original image is combined to a left portion of the shifted image, and vice versa. By horizontally shifting the image by 1/w, an image that is additionally augmented by a factor of w may be obtained.

When the second ROI is augmented as described above, the performance and stability of a prediction model may be improved even though a data set for model training is very small.

When augmenting the second ROI through the methods illustrated in FIGS. 11 and 12, the calculation module 906 may combine a first mask and a second mask generated from an additional image augmented from the same second ROI and then calculate vCDR using the first mask and the second mask. This will be described in more detail as follows.

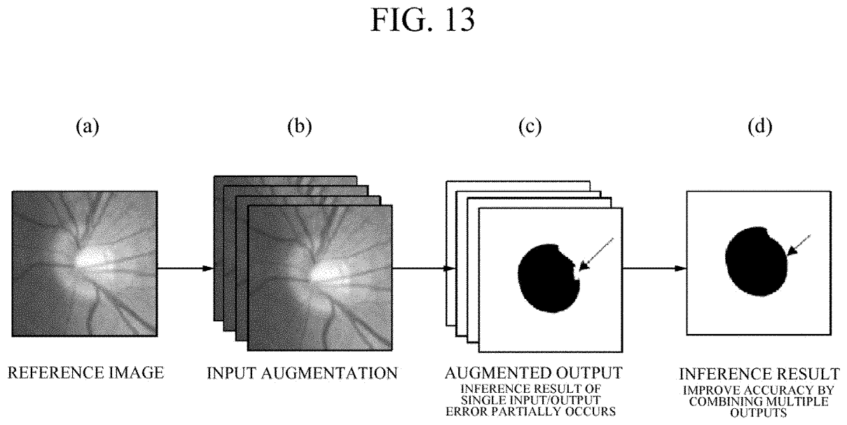

FIG. 13 is an exemplary diagram showing an example of combining a first mask and a second mask obtained by augmenting the same second ROI using the calculation module 906. In detail, FIG. 13(a) shows a second ROI, and FIG. 13(b) shows additional images augmented using the second ROI of FIG. 13(a) as a reference image. FIG. 13(c) shows a plurality of first masks generated from the additional images of FIG. 13(b). In the drawing, first masks are illustrated as an example, but the same process is applied even to second masks. Last, FIG. 13(d) shows a result of combining the plurality of first masks obtained through FIG. 13(c). In this case, the calculation module 906 inversely applies the augmentation process shown in FIG. 13(b) to the first masks obtained from FIG. 13(c) and performs combination.

As described above, in the disclosed embodiment, image augmentation is performed by horizontally or vertically moving an image. Accordingly, augmented images vary only in the position of the optic disc or the optic cup, but not in the form. Accordingly, when the first mask and the second mask obtained from the images augmented from the single second ROI are combined, a similar effect to combining multiple models can be achieved using only a single model.

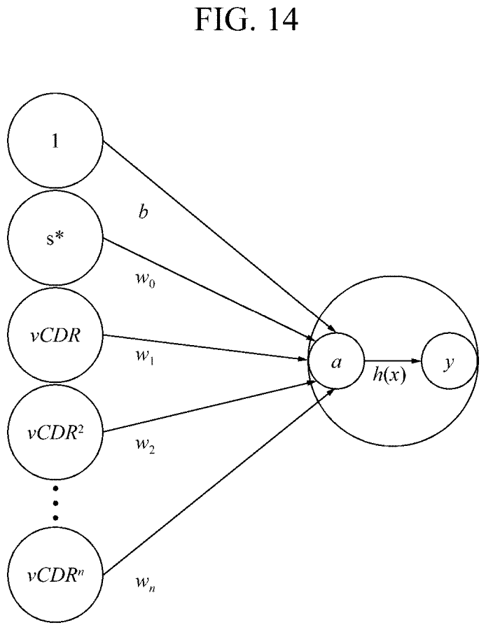

FIG. 14 is an exemplary diagram illustrating a process of determining whether glaucoma is present in an image by means of a determinator 108 according to an embodiment. As described above, the determinator 108 may determine whether glaucoma is present in the fundus image through logistic regression analysis of an image classification result of the image classification neural network 104 and a vCDR calculation result of the vCDR calculator 106. FIG. 14 shows a perceptron that indicates a multi-mode prediction model utilizing logistic regression. In this case, s, vCDR, vCDR², . . . , vCDRⁿ may be used as inputs of a logistic regression model. In this case, s indicates an image classification result (probability) using the image classification neural network 104, and vCDR, vCDR², . . . , vCDRⁿ indicate first- to nth-order terms of the vCDR calculation result (here, n is a natural number of two or more). The reason for using the multiple order terms of the vCDR is to avoid the linearity of the prediction model. The utilization of the vCDR can partially compensate for inexplicability, which is one disadvantage of a deep neural network-based prediction model, and can also improve final classification performance, thereby achieving high performance with a very small amount of data.

The disclosed embodiments may be commonly used not only in glaucoma diagnosis, but also in all medical image analysis in which the size or number of lesions is a standard of diagnosis. In particular, like a biopsy slide image captured to diagnose cancer, the disclosed embodiments can be applied in quantitatively analyzing an environment where only qualitative diagnosis is allowed because a person cannot count the number of all cells therein. In addition, in the case of age-related macular degeneration (AMD), which is diagnosed by fundus images, drusen, which are major lesions, may be observed. The disclosed embodiments may be applied in expanding modes, such as the number, positions, and area of drusen by using fundus photographs as inputs.

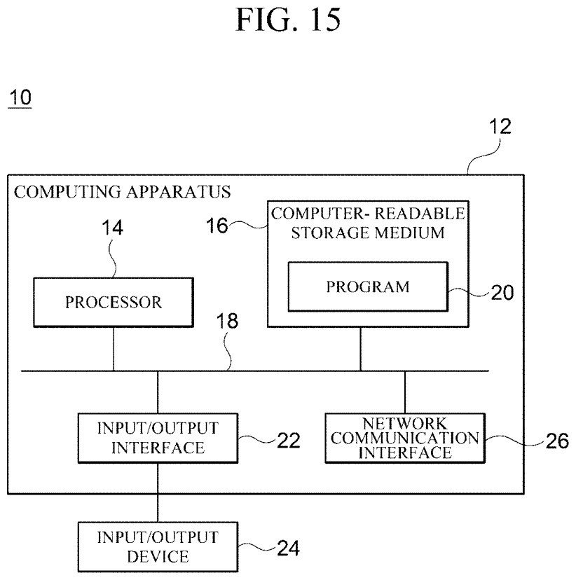

FIG. 15 is a block diagram illustrating a computing environment 10 including a computing apparatus suitable for use in example embodiments. In the illustrated embodiment, each component may have a function and capability that differ from those described below, and an additional component may be included in addition to those in the following description.

As shown, the computing environment 10 includes a computing apparatus 12. In an embodiment, the computing apparatus 12 may be a glaucoma diagnosis apparatus 100 according to embodiments of the present disclosure. The computing apparatus 12 includes at least one processor 14, a computer-readable storage medium 16, and a communication bus 18. The processor 14 may enable the computing apparatus 12 to operate according to the aforementioned example embodiment. For example, the processor 14 may execute one or more programs stored in the computer-readable storage medium 16. The one or more programs may include one or more computer-executable instructions which may be configured to enable the computing apparatus 12 to perform operations according to an example embodiment when the operations are executed by the processor 14.

The computer-readable storage medium 16 is configured to store computer-executable instructions, program codes, program data, and/or other suitable forms of information. The program 20 stored in the computer-readable storage medium 16 includes a set of instructions executable by the processor 14. In an embodiment, the computer-readable storage medium 16 may be a memory (a volatile memory such as a random access memory, a non-volatile memory, or an appropriate combination thereof), one or more magnetic disk storage devices, optical disk storage devices, flash memory devices, other forms of storage media that may be accessed by the computing apparatus 12 and are configured to store desired information, or a suitable combination thereof.

The communication bus 18 connects the processor 14, the computer-readable storage medium 16, and various other components of the computing apparatus 12 to one another.

Also, the computing apparatus 12 may include one or more input/output interfaces 22 for providing an interface for one or more input/output devices 24, and one or more network communication interfaces 26. The input/output interfaces 22 and the network communication interfaces 26 are connected to the communication bus 18. The input/output devices 24 may be connected to other components of the computing apparatus 12 through the input/output interfaces 22. The input/output devices 24 may include input devices such as a pointing device (a mouse, a trackpad, etc.), a keyboard, a touch input device (a touchpad, a touch screen, etc.), a voice or sound input device, various kinds of sensor devices, and/or a capture device and/or may include output devices such as a display device, a printer, a speaker, and/or a network card. The input/output devices 24 may be included in the computing apparatus 12 as components of the computing apparatus 12 and may be connected to the computing apparatus 12 as separate devices distinct from the computing apparatus 12.

An embodiment of the present disclosure may include a program for executing the methods described herein on a computer, and a computer-readable recording medium including the program. The computer-readable recording medium may include any one or a combination of a program instruction, a local data file, a local data structure, etc. The medium may be designed and configured specifically for the present disclosure or may be generally available in the field of computer software. Examples of the computer-readable recording medium include magnetic media such as a hard disk, a floppy disk, and a magnetic tape, optical recording media such as a CD-ROM and a DVD, and hardware devices specially configured to store and execute program instructions, such as a ROM, a RAM, and a flash memory. Examples of the program instruction may include a machine code generated by a compiler and a high-level language code that can be executed in a computer using an interpreter.

Although exemplary embodiments of the disclosure have been described in detail, it will be understood by those skilled in the art that various changes may be made without departing from the spirit or scope of the disclosure. Therefore, the scope of the present disclosure is not construed as being limited to the described embodiments but is defined by the appended claims as well as equivalents thereto.

An embodiment of the present disclosure may include a program for executing the methods described herein on a computer, and a computer-readable recording medium including the program. The computer-readable recording medium may include any one or a combination of a program instruction, a local data file, a local data structure, etc. The medium may be designed and configured specifically for the present disclosure or may be generally available in the field of computer software. Examples of the computer-readable recording medium include magnetic media such as a hard disk, a floppy disk, and a magnetic tape, optical recording media such as a CD-ROM and a DVD, and hardware devices specially configured to store and execute program instructions, such as a ROM, a RAM, and a flash memory. Examples of the program instruction may include a machine code generated by a compiler and a high-level language code that can be executed in a computer using an interpreter.

Although exemplary embodiments of the disclosure have been described in detail, it will be understood by those skilled in the art that various changes may be made without departing from the spirit or scope of the disclosure. Therefore, the scope of the present disclosure is not construed as being limited to the described embodiments but is defined by the appended claims as well as equivalents thereto.

### DESCRIPTION OF REFERENCE NUMERALS

100: glaucoma diagnosis apparatus
102: fundus image processor
104: image classification neural network
106: vertical cup-to-disc ratio (vCDR) calculator
108: determinator
202: preprocessing module
204: optic disc detection module
206: image rotation module
208: first ROI extraction module
210: second ROI extraction module
902: first mask generation module
904: second mask generation module
906: calculation module

1: A glaucoma diagnosis apparatus comprising:
a fundus image processor configured to receive a fundus image and extract a first region of interest (ROI) and a second ROI from the received fundus image;
an image classification neural network configured to learn the extracted first ROI and perform classification into a normal fundus image and a glaucoma fundus image on the basis of the learned first ROI;
a vertical cup-to-disc ratio (vCDR) calculator configured to recognize an optic disc (OD) and an optic cup (OC) from the extracted second ROI and calculate a vCDR; and
a determinator configured to aggregate a vCDR calculation result and an image classification result of the image classification neural network to determine whether glaucoma is present in the fundus image.

2: The glaucoma diagnosis apparatus of claim 1, wherein the fundus image processor comprises:
an optic disc detection module configured to detect an optic disc from the received fundus image;
an image rotation module configured to rotate the fundus image around center coordinates of the optic disc such that a slope between the center coordinates of the optic disc and a center of the fundus image is constant;
a first ROI extraction module configured to extract the first ROI from the rotated fundus image such that the detected optic disc is placed at an upper left end or an upper right end of the first ROI; and
a second ROI extraction module configured to extract the second ROI from the received fundus image such that the detected optic disc is placed at the center of the second ROI.

3: The glaucoma diagnosis apparatus of claim 2, wherein the optic disc detection module further configured to detect the optic disc using an image obtained through a polar coordinate transformation on the fundus image.

4: The glaucoma diagnosis apparatus of claim 2, wherein the fundus image processor further comprises a preprocessing module configured to resize the fundus image to a predetermined size before the optic disc is detected and horizontally flip the fundus image depending on whether the fundus image is a right fundus image or a left fundus image.

5: The glaucoma diagnosis apparatus of claim 2, wherein the image rotation module further configured to set the center of the fundus image as an origin of a coordinate plane, calculate an angle between the center coordinates of the detected optic disc and a horizontal axis (an x-axis) of the coordinate plane, and rotate the fundus image around the origin such that the angle matches a predetermined reference angle.

6: The glaucoma diagnosis apparatus of claim 5, wherein the reference angle is any one of 45 degrees or 135 degrees.

7: The glaucoma diagnosis apparatus of claim 6, wherein the image rotation module further configured to augment the number of fundus images by flipping the fundus image according to a reference line connecting the origin of the rotated fundus image and the center coordinates of the optic disc or by additionally rotating the rotated fundus image within a predetermined additional rotation range around the origin of the rotated fundus image.

8: The glaucoma diagnosis apparatus of claim 2, wherein the first ROI extraction module further configured to extract the ROI such that the center coordinates of the optic disc are spaced one-quarter of a side length from an upper left end or an upper right end of the first ROI.

9: The glaucoma diagnosis apparatus of claim 2, wherein the fundus image processor further comprises a histogram matching module configured to, when the fundus image is a test image of a machine learning model, perform histogram matching on the first ROI of the test image on the basis of an average histogram of images included in a learning dataset of the machine learning model.

10: The glaucoma diagnosis apparatus of claim 2, wherein the vCDR calculator comprises:
a first mask generation module configured to generate a first mask corresponding to the optic disc using an image obtained through a polar coordinate transformation on the second ROI;
a second mask generation module configured to extract a sub-region corresponding to the first mask from the second ROI and generate a second mask corresponding to the optic cup from the extracted sub-region; and
a calculation module configured to overlay the first mask and the second mask and calculate the vCDR.

11: The glaucoma diagnosis apparatus of claim 10, wherein the first mask generation module further configured to augment the number of second ROIs by horizontally or vertically moving the center of the second ROI within a predetermined range or by horizontally shifting the image obtained through the polar coordinate transformation on the second ROI within a predetermined range.

12: The glaucoma diagnosis apparatus of claim 11, wherein the calculation module further configured to calculate the vCDR using a result of combining the first mask and the second mask generated from an additional image generated by augmenting the same second ROI.

13: The glaucoma diagnosis apparatus of claim 1, wherein the determinator further configured to determine whether glaucoma is present in the fundus image through logistic recession analysis on the image classification result and the vCDR calculation result.

14: The glaucoma diagnosis apparatus of claim 13, wherein the determinator further configured to perform the logistic regression analysis by using first- to nth-order terms (here, n is a natural number of 2 or more) of the vCDR calculation result as an input value in addition to the image classification result.
